(19) United States
(12) Patent Application Publication
(10) Pub. No.: US 2021/0386462 A1
(43) Pub. Date: Dec. 16, 2021

(54) WRIST IMPLANT

(71) Applicant: David V. TUCKMAN, Manhasset, NY (US)

(72) Inventor: David V. TUCKMAN, Manhasset, NY (US)

(21) Appl. No.: 17/212,153

(22) Filed: Mar. 25, 2021

Related U.S. Application Data
(60) Provisional application No. 63/037,233, filed on Jun. 10, 2020.

(51) Int. Cl. A61B 17/72; A61B 17/80

(52) U.S. Cl. CPC A61B 17/7291; A61B 17/8061

(57) ABSTRACT

A wrist implant comprising a body section wherein the body section is elongated, wherein the body section has a first end and a distal end along the longitudinal extension of the body section with an elongated opening positioned adjacent to the first end in the body section, at least one wing section coupled to the body section and extending substantially transverse to the elongated extension of the body section, at least one opening in said elongated extension, and at least one opening positioned in the distal end of the body section.

### CROSS REFERENCE TO RELATED APPLICATIONS

This application is a non-provisional application that claims priority to provisional application Ser. No. 63/037,233 filed on Jun. 10, 2020, the disclosure of which is hereby incorporated herein by reference.

### BACKGROUND

One embodiment of the invention relates to a wrist implant that is configured to bridge between two different parts of a wrist. The wrist joint includes many different bones including but not limited to the capitate, the hamate, the lunate, the scaphoid, the trapezium, the trapezoid, the pisiform, and the triquetrum. One bone in particular sits towards the center of the wrist joint, which is the capitate. The capitate is one of the largest bones of the wrist. It is situated between the hamate and the trapezoid in the distal row of carpal bones and sits between the ulna and the radius. It also articulates with the third metacarpal. When this bone is injured it is important to stabilize the bone and the joint in this region. Therefore, there is a need for a plate or implant that can be used to stabilize a wrist joint in an efficient manner.

### SUMMARY

In at least one embodiment there is a wrist implant comprising a body section of a plate, wherein the body section is elongated, and wherein the body section has a first end and a distal end along the longitudinal extension of the body section. There is also an elongated opening positioned adjacent to the first end in the body section. Extending off of this body section is at least one wing section coupled to the body section and which extends substantially transverse to the elongated extension of the body section. There is at least one opening in said elongated extension and at least one opening positioned in the distal end of the body section.

In at least one embodiment the body section extends along a plane.

In at least one embodiment there is at least one additional opening extending substantially transverse to the plane of the body.

In at least one embodiment, the device further comprises at least one additional opening positioned in the body.

In at least one embodiment, at least one additional opening is positioned adjacent to the distal end of the body section.

In at least one embodiment, at least one additional wing section is coupled to the body section.

In at least one embodiment, at least one hole is positioned in at least one additional wing section.

In at least one embodiment, at least one opening positioned in the distal section is angled substantially transverse to an extension of the plane of the body section.

In at least one embodiment, at least one opening in said distal end is beveled.

In at least one embodiment, at least one elongated opening is beveled.

In at least one embodiment the plate or body is configured to be coupled to at least one of the capitate, the hamate, the lunate and the triquetrum.

Generally, the bones of the wrist joint include the scaphoid, the lunate, the pisiform, the hamate, the capitate, the trapezoid, and the trapezium. When a wrist is broken or is severely strained a plate may need to be inserted to support the healing of the bones in this wrist joint. One benefit of this plate is that it is designed to join one or more of these bones together with a stabilizing plate to allow for healing in a particular bone or in the wrist joint overall. In particular, this plate is configured to couple the capitate together with the adjacent bones such as the hamate, the lunate and triquetrum to heal the wrist joint, including the capitate bone.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 is a top plan view of the implant;

FIG. 2 is a top shaded view of the implant;

FIG. 3 is a side view of the implant;

FIG. 4 is a perspective view of the implant;

FIG. 5 is an end view of the implant;

FIG. 6A is a first view of the implant coupled to a wrist joint; and

FIG. 6B is another view of the implant coupled to the wrist joint.

FIG. 7A is a substantially solid view of the plate mounted in the wrist joint from a bottom view; and

FIG. 7B is a substantially three-dimensional view of the plate mounted in the wrist joint.

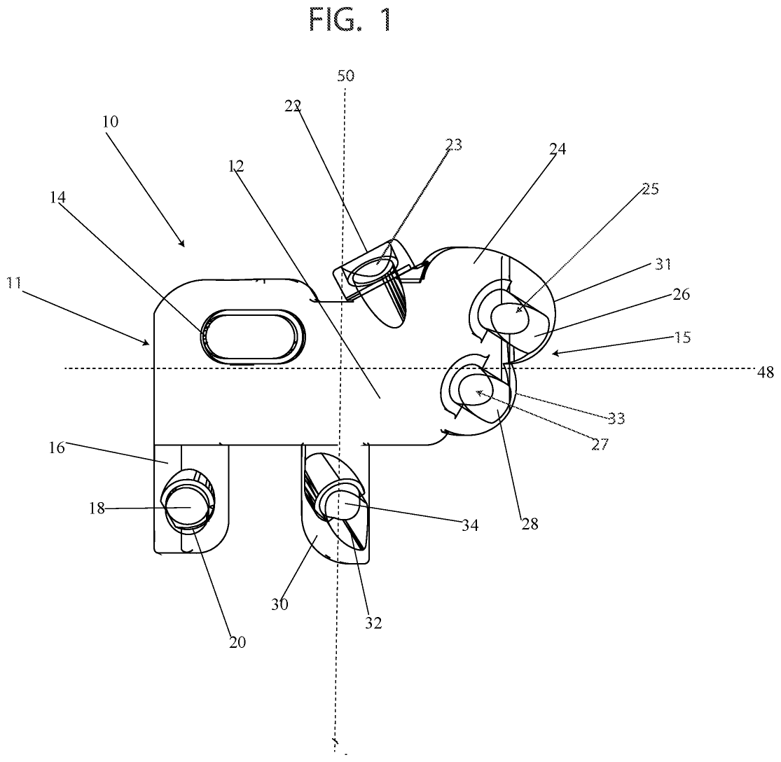

### DETAILED DESCRIPTION

Referring in detail to the drawings, there is disclosed a plate for securing a wrist joint wherein the plate 10 includes a body section 12, as well as a plurality of holes. the body section 12 includes a substantially planar plate which is comprised of a substantially flat surface. This planar plate 10 extends along a plane, wherein a first end 11 of the body section 12 is on a first side of the latitudinal axis and a second end or distal end 15 of the body section 12 is on the opposite side of the latitudinal axis. The body section 12 extends along a longitudinal axis 48 and has a latitudinal axis 50 as well. A longitudinal hole or opening 14 extends along the longitudinal axis in a substantially oval manner such that it has a longer longitudinal extension than latitudinal extension. This geometry allows for some tolerance for movement when initially installing the plate with screws. This hole or opening has a surrounding rim 17. This rim has a beveled edge which is configured to receive a screw. Extending off of the plate 10 are a plurality of tabs 16 and 30 which are each configured to house holes or openings 18 and 34. Each of these tabs 16 and 30 extend transverse to the longitudinal extension or along the latitudinal axis 50. There is at least one hole 18 which is positioned in tab 16, and at least one hole 34 positioned in tab 30. Each of these holes has a surrounding elevated section. For example, hole 18 has an elevated rim 20 which extends as a substantially cylindrical region around hole 18. In addition, hole 34 has an elevated rim 32 which extends around the hole 34. These elevated rims are configured as cylindrical and which in at least one instance can be angled offset from a full 90-degree intersection of a plane of the plate surface. For example, the extension of this elevated rim opening can be offset from transverse from the plane of extension of the plate 10. For example, this substantial offset can be 30 degrees offset, 45 degrees offset of 60 degrees offset from transverse or perpendicular to the plane of the plate. One reason for the angling of these elevated rims is to allow for angled direction of screws which can be guided in the rims to connect two particular bones together.

In addition, there is another opening 23 which has a cylindrical rim 22 extending out therefrom. This opening 23, extends laterally along transverse to the side of the plane of plate 10. For example, this cylindrical rim extends at a substantially perpendicular angle to the extension of the plate 10. In at least one embodiment the extension of this opening 23 is offset from perpendicular by at least 10 degrees. In another embodiment the orientation of opening 23 is offset from perpendicular by at least 30 degrees. The cylindrical rim 22 formed around opening 23 has a full 360-degree rim for enclosure. In addition, there are two other holes or openings 25 and 27 formed in a distal region or distal end 24 of the plate 10. Hole or opening 25 has a cylindrical rim 26 formed around it wherein this hole and the corresponding cylindrical rim 26 extends along an axis that is offset from perpendicular to the plane of the plate. In addition, hole 27 and corresponding cylindrical rim 28 extending along an axis that is offset from perpendicular to the plane of the plate as well. This plate has ends 31 and 33 which are rounded and which form extensions for receiving the holes.

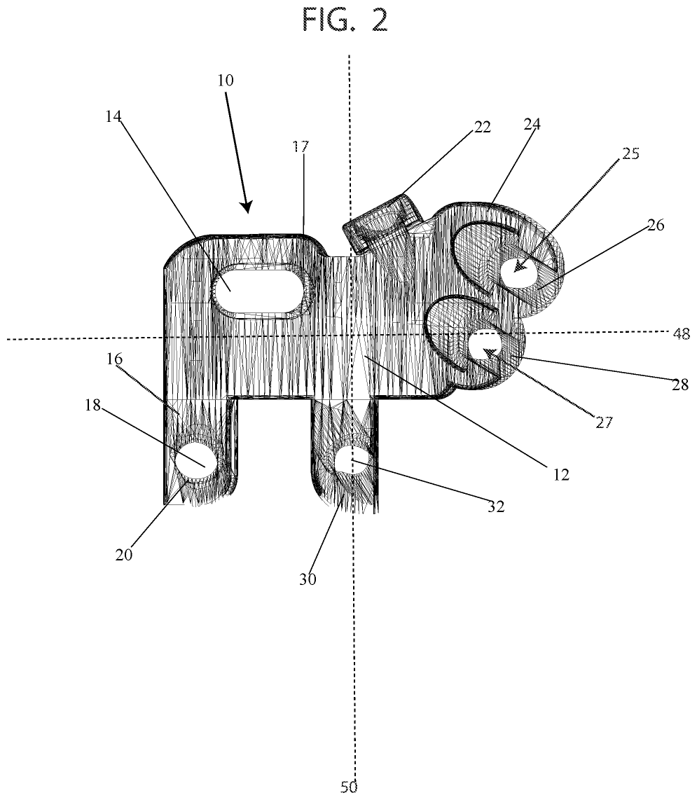

FIG. 2 shows a shaded view of this plate which then shows the cylindrical rims surrounding the corresponding holes as well.

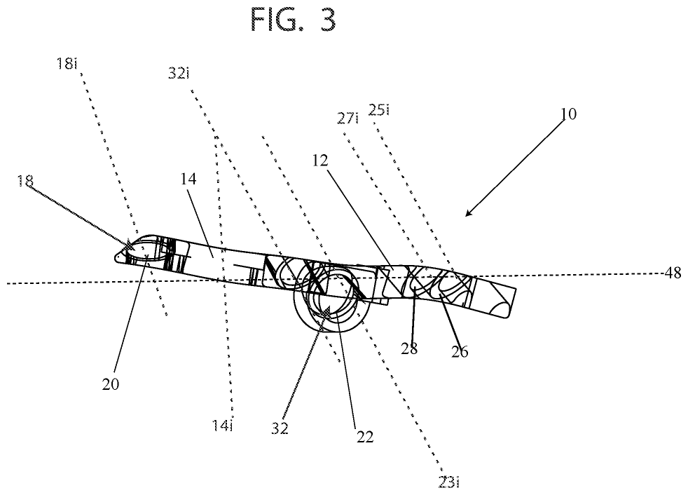

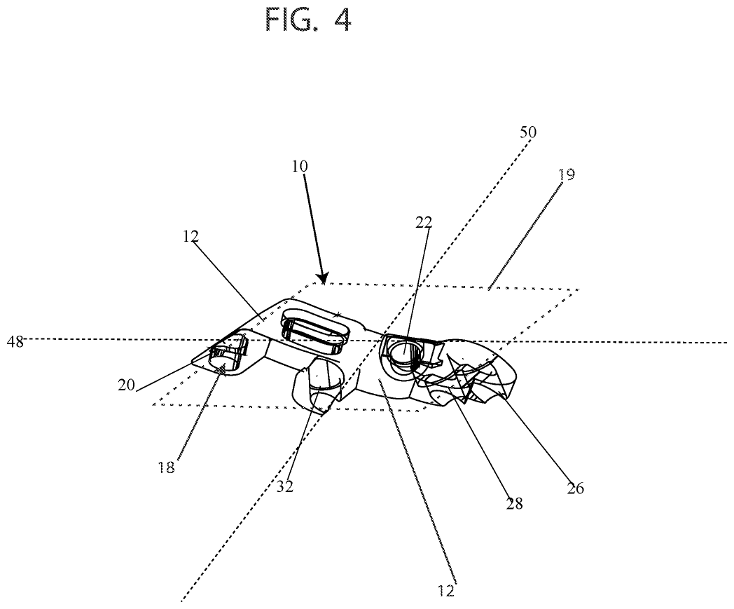

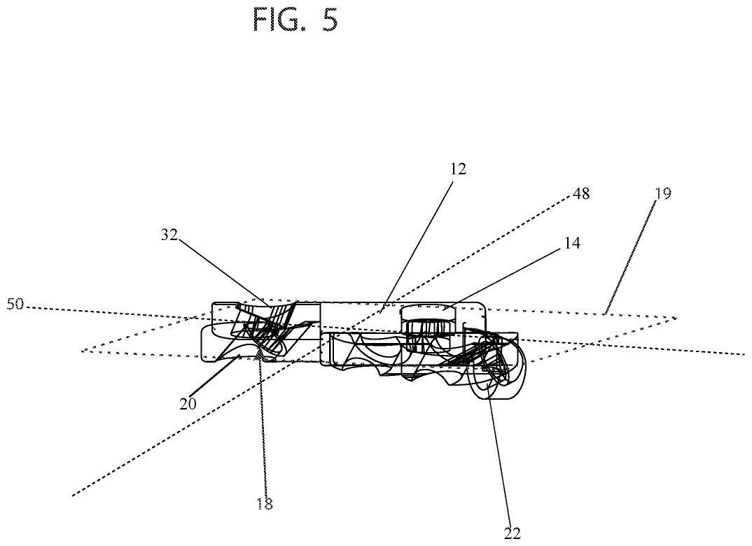

FIG. 3 shows the different axes of extension through the corresponding holes in the plate. For example, there is a first axis of extension 18i through hole 18, another axis of extension 14i through hole or opening 14. The axis of extension 18i is offset at least partially from a perfectly perpendicular angle. The axis of extension 14i of hole 14 is substantially perpendicular to the planar extension of plate 10. The axis of extension 23i of hole or opening 23 is substantially transverse to the planar extension of plate 10 (see planar extension 19 in FIGS. 4 and 5). The axis of extension 27i of hole 27 is substantially or at least partially offset from perpendicular to the planar extension of plate 10. The axis of extension 25i of hole 25 is also at least partially offset from perpendicular to the planar extension of plate 10 as well.

FIG. 4 shows the plane or planar extension 19 showing the planar extension of plate 10 having body 12. In this plate there are shown holes or openings 14, 18, 23, 25, 27 and 34. In addition there are also shown dashed lines showing a longitudinal axis 48 and latitudinal axis 50 as well.

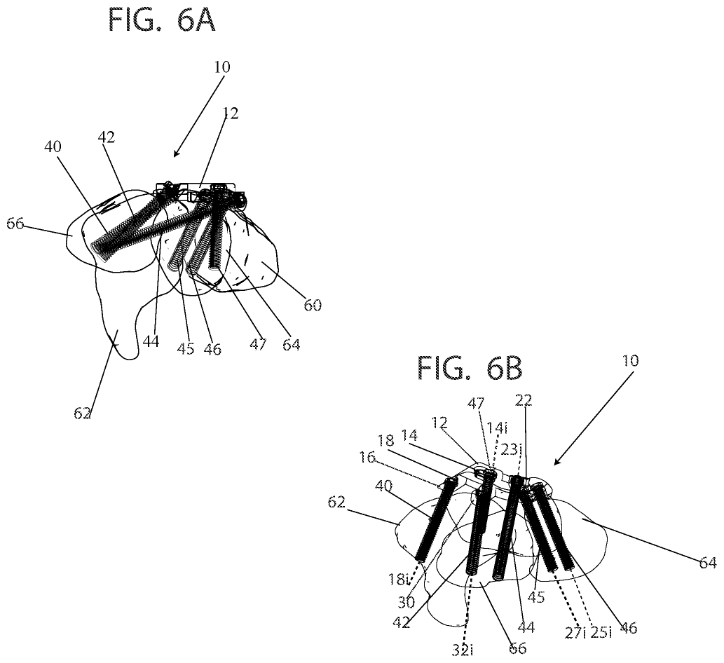

FIG. 5 shows the plane 19 which shows the planar extension of plate 10 having body section 12. As indicated this plate has openings or holes 14, 18, 23, 25, 27 and 34. In addition, the plane 19 extends along longitudinal axis 48, and latitudinal axis 50. FIGS. 6A and 6B show the plate used as an implant to secure bones in a person's wrist. For example, a plurality of screws are configured to secure the plate 10. These screws include screws 40, 42, 44, 45, 46, and 47. The screws are configured to secure the plate 10 to a plurality of bones in a user's wrist. For example, there are shown a series of bones 60, 62, 64, and 66. The screws such as screws 40, 42, 44, 45, 46 and 47 are configured to extend along the axes of extension of the associated rims. For example, screw 40 extends substantially along axis or extension 18i, screw 42 extends substantially along axis 32i. Screw 44 extends substantially along axis 23i, screw 45 extends substantially along axis 27i and screw 46 extends along axis 25i and screw 47 extends along axis extension 14i.

In this view there is the capitate 60 and the hamate 62 shown secured by the plate while there is also shown other bones such as the lunate 64 and the triquetrum 66 which are configured to also be secured to the plate.

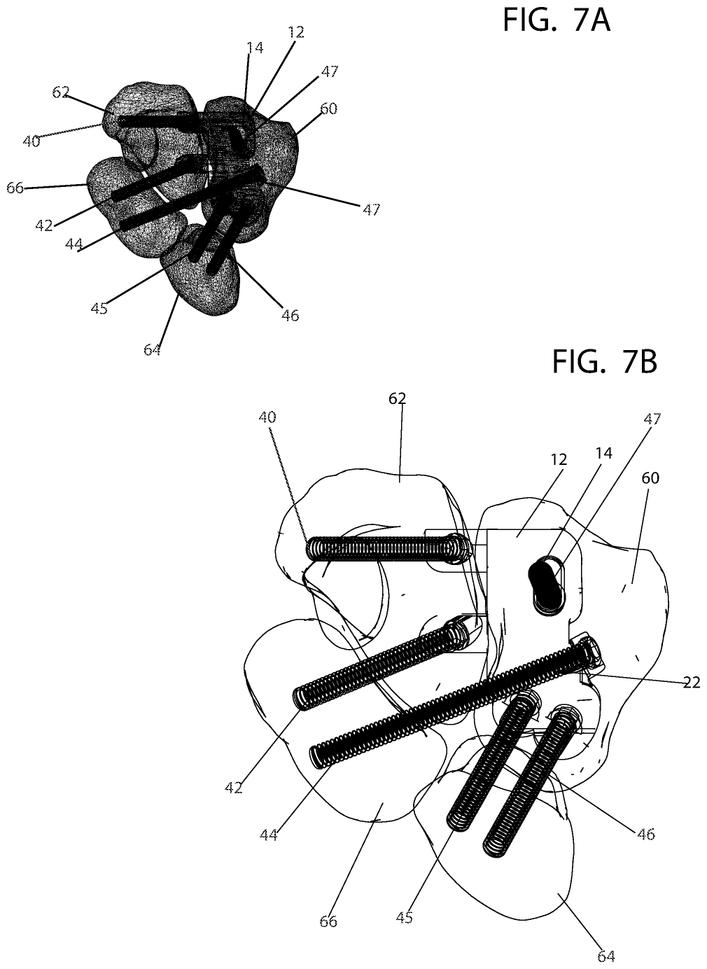

FIGS. 7A and 7B show more clearly the joining features of the plate 10 which are configured to join the capitate 60 with the hamate 62, the lunate 64 and the triquetrum 66. As shown screw 40 connects plate body section 12 to the hamate. Screw 44 is configured to connect the plate 10 and plate body section 12 on the capitate 60 with the hamate 62 and the triquetrum 66. Screws 45 and 46 are configured to connect the plate body section 12 on the capitate 60 to the lunate 64. Screw 47 is configured to connect the plate body or body section 12 with the capitate 60. Thus, the angles of the openings result in angled orientation of the selective screws to allow for the joining of plate 10 with these bones in the wrist joint to stabilize the capitate in the event of an injury such as a broken capitate bone.

Accordingly, while at least one embodiment of the present invention has been shown and described, it is obvious that many changes and modifications may be made thereunto without departing from the spirit and scope of the invention.

What is claimed is:

1. A wrist implant comprising:
a body section wherein the body section is elongated, wherein the body section has a first end and a distal end along the longitudinal extension of the body section;
an elongated opening positioned adjacent to the first end in the body section;
at least one wing section coupled to the body section and extending substantially transverse to the elongated extension of the body section;
at least one opening in said elongated extension; and
at least one opening positioned in the distal end of the body section.

2. The wrist implant as in claim 1, wherein the body section extends along a plane.

3. The wrist implant as in claim 2, further comprising at least one additional opening extending substantially transverse to the plane of the body.

4. The wrist implant as in claim 1, further comprising at least one additional opening positioned in the body section.

5. The wrist implant as in claim 1, wherein said at least one additional opening is positioned adjacent to the distal end of the body section.

6. The wrist implant as in claim 1, further comprising at least one additional wing section coupled to the body section.

7. The wrist implant as in claim 6, further comprising at least one hole positioned in said at least one additional wing section.

8. The wrist implant as in claim 2, wherein said at least one opening positioned in the distal section is angled substantially transverse to an extension of the plane of the body section.

9. The wrist implant as in claim 1, wherein said at least one opening in said distal end is beveled.

10. The wrist implant as in claim 1, wherein said at least one elongated opening is beveled.

11. The wrist implant as in claim 1, wherein the body section is configured to be coupled to a capitate bone.

12. The wrist implant as in claim 1, wherein the body section is configured to be coupled to the hamate.

13. The wrist implant as in claim 1, wherein the body section is configured to be coupled to the lunate.

14. The wrist implant as in claim 1, wherein the body section is configured to be coupled to the triquetrum.

15. The wrist implant as in claim 1, wherein the body section is configured to couple the capitate to the hamate.

16. The wrist implant as in claim 1, wherein the body section is configured to couple the capitate to the lunate.

17. The wrist implant as in claim 1, wherein the body section is configured to couple the capitate to the triquetrum.

18. The wrist implant as in claim 1, wherein the body section is configured to couple the capitate to the hamate, the lunate and the triquetrum.
